(19) United States
(12) Patent Application Publication
(10) Pub. No.: US 2006/0251744 A1
(43) Pub. Date: Nov. 9, 2006

(54) Method for preparing ginkgo extracts having a reduced content of polycyclic aromatic hydrocarbons

(75) Inventors: Rainer Oschmann, Landau (DE); Frank Waimer, Karlsruhe (DE); Hermann Hauer, Karlsruhe (DE)

Correspondence Address: EDWARDS & ANGELL, LLP, P.O. BOX 55874, BOSTON, MA, 02205, US

(73) Assignee: Bioplanta Arzneimittel GmbH, Ettlingen (DE)

(21) Appl. No.: 11/416,769

(22) Filed: May 2, 2006

(30) Foreign Application Priority Data
May 3, 2005 (DE) 10 2005 020 640.9

(51) Int. Cl. A61K 36/16

(52) U.S. Cl. 424/752

(57) ABSTRACT

The present invention relates to an improved multi-step method for preparing an extract from Ginkgo biloba having a reduced content of polycyclic aromatic hydrocarbons. The invention further relates to an extract from Ginkgo biloba having a reduced content of polycyclic aromatic hydrocarbons which is obtainable by the method according to the present invention, as well to its use.

The present invention relates to an improved multi-step method for preparing an extract from Ginkgo biloba having a reduced content of polycyclic aromatic hydrocarbons. The invention further relates to an extract from Ginkgo biloba having a reduced content of polycyclic aromatic hydrocarbons, which is obtainable by the method according to the present invention, as well as to its use.

Since decades, extracts from the leaves of Ginkgo biloba are used as a medicament. They are currently used for the treatment of different kinds of dementia and symptoms thereof as well as cerebral and peripheral blood circulation disorders. Ingredients, the efficacy is associated with, are terpene lactones (ginkgolides A, B, C und bilobalide) as well as glycoside and flavones (quercetin, kaempferol and isorhamnetin). However, the leaves of Ginkgo biloba also contain considerable amounts of components which do not contribute to the desired efficacy, but which may be responsible for risks and side effects. These are particularly unpolar plant ingredients such as ginkgolic acids and unpolar impurities due to environmental influences such as polycyclic aromatic hydrocarbons (PAHs). In a Ginkgo extract which is efficacious and at the same time as safe as possible and as low in side effects as possible, these compounds should thus not be present to the largest possible extent.

Due to the existing or increasing atmospheric pollution in large parts of the world, which is a consequence of the rapidly increasing consumption of fossil fuels such as petroleum and which also concerns growing areas of Ginkgo biloba, Ginkgo leaves are recently provided to an increasing extent, which are polluted with considerable amounts of unpolar impurities due to environmental influences, particularly polycyclic aromatic hydrocarbons (PAHs). In this regard, PAHs are a general term for aromatic compounds having fused ring systems such as fluorene, phenanthrene, anthracene, fluoranthene, pyrene, benz[a]anthracene, chrysene, benzo[b]fluoranthene, benzo[k]fluoranthene, benzo[a]pyrene, indeno[1,2,3-cd]pyrene, dibenzo[ah]anthracene and benzo[ghi]perylene. At least a part of the PAHs are carcinogenic such that there is a significant necessity to ensure that extracts produced from polluted Ginkgo leaves are set free from these pollutants to the largest possible extent. Generally, in the case of carcinogenic substances, a lower limit, under which PAHs are considered to be harmless, cannot be defined.

A Ginkgo extract which has a low content of Ginkgolic acid (<10 ppm and <1 ppm, respectively) is already described in EP 431535 B1. However, it is not described whether this method is simultaneously capable of largely depleting PAHs present in Ginkgo leaves. It has now been found that the method according to EP 431535 B1 already leads to a PAH depletion. Since there is no limit of harmlessness, there is still a significant necessity to further improve this method such that a higher depletion of PAHs occurs.

Therefore, it is the object underlying the present invention to modify the method described in EP 431535 B1 such that the contents of PAHs are further minimized. Furthermore, subject of the present invention are also Ginkgo extracts, which are obtainable according to this modified method as well as their use.

According to claim 3 of EP 431535 B1 (the preferred parameters of claim 4 depending thereon are given in parenthesis), the total disclosure content of which shall be explicitly incorporated into the present application by reference, the method for preparing an extract from Ginkgo biloba leaves claimed therein is characterized in that

(a) fresh or dried green leaves of Ginkgo biloba are extracted at a temperature of about 40 to 100° C. using aqueous acetone, an aqueous alkanol having 1 to 3 carbon atoms or anhydrous methanol,

(b) the organic solvent is largely separated from the extract to a maximum content of 10% by weight (maximum content of 5% by weight), wherein water may be added during the final distillation steps,

(c) the remaining concentrated aqueous solution is diluted with water to a solids content of 5 to 25% by weight (15 to 20% by weight), followed by cooling to a temperature below 25° C. (cooled to a temperature of about 10 to 12° C.) and allowing to stand until a precipitate is formed, and the resulting precipitate, which consists of the lipophilic components that are not well soluble in water, is removed,

(d) ammonium sulfate is added to the remaining aqueous solution (to a content of 30% by weight) and the solution formed is extracted with methyl ethyl ketone or a mixture of methyl ethyl ketone and acetone (in a ratio of 9:1 to 4:6, preferably 6:4),

(e) the extract obtained is concentrated to a solids content of 50 to 70% and the concentrate thus obtained is diluted with water such that a solution containing 50% by weight water and 50% by weight ethanol at a solids content of 10% by weight is obtained,

(f) an aqueous solution of a lead salt (lead acetate, lead hydroxide acetate or lead nitrate or an aqueous suspension of lead hydroxide) is added to the solution thus obtained until a change in colour from brown to amber occurs, and the precipitate formed is removed, or a polyamide is used instead of the lead salt,

(g) the remaining aqueous-alcoholic solution is extracted with an aliphatic or cycloaliphatic solvent having a boiling point of 60 to 100° C. in order to further remove the alkylphenol compounds,

(h) the remaining aqueous-alcoholic solution is concentrated under reduced pressure to an ethanol content of about 5% and ammonium sulfate is added to a content of 20% by weight,

(i) the solution obtained is extracted with a mixture of methyl ethyl ketone and ethanol in a ratio of 8:2 to 5:5, preferably 6:4,

(k) the resulting organic phase is concentrated to a solids content of 50 to 70% by weight,

(l) the resulting concentrate is concentrated under reduced pressure at a maximum temperature of 60 to 80° C., thereby obtaining a dry extract having a water content of less than 5%.

It has now surprisingly been found that a better depletion of PAHs can be achieved by a combination of several modifications of the method according to EP 431535 B1 than it is obtained when example 1 of EP 431535 B1 is reproduced.

According to the present invention, the following modifications contribute to a more effective PAH depletion:
in step (b), the organic solvent is separated to a maximum content of 2% by weight, preferably 1% by weight,
in step (c) cooling is carried out to ≦6° C.,
in step (c) the period for the precipitate formation is additionally extended to at least 1 hour, preferably to at least 10 hours at ≦6° C.,
after step (e) a filtration step is incorporated and the filtrate is further processed,
in step (g) heptane is employed as the aliphatic or cycloaliphatic solvent,
in step (g) the extraction using heptane is carried out at least five times,
in step (k) drying is carried out with up to 20% by weight ammonium sulfate prior to concentrating,
after step (k) ethanol is added such that an ethanol content of at least 80% by weight results and the temperature of up to 12° C. is maintained for 2 to 10 h and a filtration is carried out.

Thus, the method according to the present invention for preparing an extract from Ginkgo biloba leaves is characterized in that

(a) fresh or dried green leaves of Ginkgo biloba (drug) are extracted at a temperature of about 40 to 100° C., preferably 40 to 60° C. with aqueous acetone having a content of 20-90% by weight, an aqueous alkanol having 1 to 3 carbon atoms and a content of 20-90% by weight (methanol, ethanol, n-propanol, isopropanol) or anhydrous methanol, wherein the drug-to-solvent ratio is 1:4 to 1:20, preferably 1:5 to 1:10,

(b) the organic solvent is largely separated from the extract to a maximum content of 2% by weight, preferably 1% by weight, wherein water may be added during the final distillation steps,

(c) the remaining concentrated aqueous solution is diluted with water to a solids content of 5 to 25% by weight, cooled under agitation to a temperature below 6° C. and allowed to stand for at least 1 hour, preferably for at least 10 hours at this temperature and the resulting precipitate consisting of lipophilic components which are not well soluble in water, is removed,

(d) ammonium sulfate (preferably about 30% by weight) is added to the remaining aqueous solution and the solution formed is extracted with methyl ethyl ketone or a mixture of methyl ethyl ketone and acetone (in a ratio of preferably 6:4),

(e) the extract obtained is concentrated to a solids content of 50 to 70% and the concentrate thus obtained is diluted with water and ethanol such that a solution containing 50% by weight water and 50% by weight ethanol at a solids content of 10% is obtained,

a filtration is carried out and the filtrate is further processed in (f),

(f) an aqueous solution of a lead salt (preferably lead hydroxide acetate) is added to the solution thus obtained, until a change in colour from brown to amber occurs, and the precipitate formed is removed, or a polyamide is used instead of the lead salt,

(g) the remaining aqueous alcoholic solution is extracted with heptane, wherein the extraction is carried out at least five times in order to further remove the alkylphenol compounds,

(h) the remaining aqueous alcoholic solution is concentrated to an ethanol content of about 5% under reduced pressure and ammonium sulfate is added to a content of 20% by weight,

(i) the solution obtained is extracted with a mixture of methyl ethyl ketone and ethanol in a ratio of 8:2 to 5:5, preferably 6:4,

(k) the resulting organic phase is dried with ≦20% by weight ammonium sulfate and concentrated to a solids content of 50 to 70% by weight,

added with ethanol such that an ethanol content of at least 80% by weight is obtained, and maintained for at least 2 h, preferably up to 10 h at ≦12° C. and filtered,

(l) the resulting filtrate is concentrated under reduced pressure at a maximum temperature of 60 to 80° C. and dried, thereby obtaining a dry extract having a water content of less than 5%.

The preferred extraction solvent in step (a) is aqueous acetone, particularly preferred with an acetone content of about 60% by weight.

A further subject of the present invention are extracts, particularly dry extracts which are obtainable by the method according to the present invention and which are characterized by having a reduced content of PAHs compared to the corresponding extract according to EP 431535 B1.

According to the European Pharmacopoeia dry extracts generally have a dry residue of at least 95% by weight.

The extracts according to the present invention can be administered in the form of powders, granules, tablets, dragées (coated tablets) or capsules, preferably orally. In order to prepare tablets, the extract is mixed with suitable pharmaceutically acceptable adjuvants such as lactose, cellulose, silicon dioxide, croscarmellose and magnesium stearate and pressed into tablets which are optionally provided with a suitable coating made of, for example, hydroxymethylcellulose, polyethyleneglycol, pigments (such as titanium dioxide, iron oxide) and talcum. The extract according to the present invention can also be filled into capsules, optionally under the addition of adjuvants such as stabilizers, fillers and the like. The dosage is such that 10 to 2000 mg, preferably 50 to 1000 mg and particularly preferred 100 to 500 mg extract are administered per day.

Furthermore, subject of the present invention are medicaments, food products and other preparations, which contain these extracts, optionally in combination with other substances such as active ingredients and/or pharmaceutically acceptable adjuvants. The term “food product” as used herein particularly refers to dietetic food products, dietary supplement products as well as medical food and dietary supplement.

### EXAMPLES

### Comparative Example 1

Dried and ground leaves of Ginkgo biloba with a PAH contamination due to environmental influences were extracted twice at a temperature of about 58° C. using each time 7.5 times their weight (w/w) made up of acetone/water 60/40 (w/w) (step a)).

The organic solvent was largely separated from the combined extract solutions, wherein water was added (solids content: about 15% by weight; acetone content: 2.51%; step b)). The product was cooled to a temperature of about 12° C. under agitation and after one hour the resulting precipitate is removed (step c)).

About 30% by weight ammonium sulfate was added to the remaining aqueous solution and the solution formed was extracted with a mixture of methyl ethyl ketone and acetone in a ratio of 6:4 (w/w) (step d)).

The extract thus obtained was concentrated to a solids content of about 60% by weight and the concentrate thus obtained was diluted with water and ethanol such that a solution containing 50% by weight water and 50% by weight ethanol at a solids content of about 10% was obtained (step e)). This solution was added with an aqueous solution of lead hydroxide acetate and the precipitate formed was separated (step f)).

The remaining aqueous alcoholic solution was extracted three times using each time ⅓ of its volume made up of hexane (step g)).

Then the remaining aqueous alcoholic solution was concentrated under reduced pressure (ethanol content about 5%) and about 20% by weight ammonium sulfate was added (step h)).

The solution obtained was extracted with a mixture of methyl ethyl ketone and ethanol in a ratio of 6:4 (w/w) (step i)).

The resulting organic phase was dried with about 20% by weight ammonium sulfate and concentrated to a solids content of about 60% by weight (step k)). The concentrate was freeze-dried (step I)).

### Example 1

Dried and ground leaves of Ginkgo biloba with a PAH contamination due to environmental influences (from the same batch as in Comparative Example 1) were extracted twice using each time 7.5 times their weight (w/w) made up of acetone/water 60/40 (w/w) at a temperature of about 58° C. (step a)).

The organic solvent was largely removed from the combined extract solution, wherein water was added (solids content: about 15% by weight; acetone content <0.01%) (step b)).

The solution was cooled to a temperature of about 4° C. under agitation and after one hour the precipitate formed was removed (step c)).

About 30% by weight ammonium sulfate was added to the remaining aqueous solution and the solution formed was extracted with a mixture of methyl ethyl ketone and acetone in a ratio of 6:4 (w/w) (step d)).

The extract obtained was concentrated to a solids content of about 60% by weight and the concentrate thus obtained was diluted with water and ethanol such that a solution containing 50% by weight water and 50% by weight ethanol at a solids content of about 10% by weight was obtained. The solution was filtered (step e)), the filtrate was added with an aqueous solution of lead hydroxide acetate and the precipitate formed was separated (step f)). The remaining aqueous alcoholic solution was extracted three times using each time ⅓ of its volume made up of heptane (step g)).

Then the remaining aqueous alcoholic solution was concentrated under reduced pressure (ethanol content of about 5%) and about 20% by weight ammonium sulfate was added (step h)).

The solution obtained was extracted with a mixture of methyl ethyl ketone and ethanol in a ratio of 6:4 (w/w) (step i)).

The resulting organic phase was dried with about 20% by weight ammonium sulfate and concentrated to a solids content of about 60% by weight. The concentrate was added with ethanol such that an ethanol content of at least 80% by weight was obtained. The product was cooled to 10° C. for five hours, filtered (step k)) and freeze-dried (step I)).

### Example 2

Dried and ground leaves of Ginkgo biloba with a PAH contamination due to environmental influences (from the same batch as in Comparative Example 1) were extracted twice at a temperature of about 58° C. using each time 7.5 times their weight (w/w) made up of acetone/water 60/40 (w/w) (step a)).

The organic solvent was largely separated from the combined extract solutions, wherein water was added (solids content: about 15% by weight; acetone content: 0.01%, step b)).

The product was cooled to a temperature of about 4° C. under agitation and after about 15 h the resulting precipitate was removed (step c)).

About 30% by weight ammonium sulfate was added to the remaining aqueous solution and the solution formed was extracted with a mixture of methyl ethyl ketone and acetone in a ratio of 6:4 (w/w) (step d)).

The extract obtained was concentrated to a solids content of about 60% by weight and the concentrate thus obtained was diluted with water and ethanol such that a solution containing 50% by weight water and 50% by weight ethanol at a solids content of about 10% by weight was obtained. The solution was filtered (step e)), the filtrate was added with an aqueous solution of lead hydroxide acetate and the precipitate formed was separated (step f)).

The remaining aqueous alcoholic solution was extracted five times using each time ⅓ of its volume made up of heptane (step g)).

Then the remaining aqueous alcoholic solution was concentrated under reduced pressure (ethanol content of about 5%) and about 20% by weight ammonium sulfate was added (step h)).

The solution obtained was extracted with a mixture of methyl ethyl ketone and ethanol in a ratio of 6:4 (w/w) (step i)).

The resulting organic phase was dried with about 20% by weight ammonium sulfate and concentrated to a solids content of about 60% by weight. The concentrate was added with ethanol, such that an ethanol content of at least 80% by weight was obtained. The product was cooled to 10° C. for five hours, filtered (step k)) and freeze-dried (step I)).

Results:

The contents of the polycyclic aromatic hydrocarbons (PAH) fluorene, fluoranthene, pyrene and benzo[k]fluoranthene in the resulting extracts can be seen from the following table. It is found that the sum of the contents in Examples 1 and 2 according to the present invention is significantly lower than in Comparative Example 1. Furthermore, a still lower value is found in Example 2 than in Example 1 due to additional modifications of the method.

TABLE 1
Compositions of the extracts according to the above examples

| | Comparative | | |
|---|---|---|---|
| Extract according to | Example 1 | Example 1 | Example 2 |
| acetone content (step b)) | 2.51% | <0.01% | 0.01% |
| temperature (step c)) | 12° C. | 4° C. | 4° C. |
| time of precipitation (step c)) | 1 h | 1 h | about 15 h |
| Filtration (step e)) | no | yes | yes |
| Extraction solvent (step g)) | Hexane | Heptane | Heptane |
| Extraction (step g)) | 3 times with | 3 times with | 5 times with |
| | ⅓ vol. | ⅓ vol. | ⅓ vol. |
| Precipitation (step k)) | no | yes | yes |
| Fluorene [ppb] | 1.6 | 1.9 | 1.9 |
| Fluoranthene [ppb] | 3.2 | 3.5 | 2.4 |
| Pyrene [ppb] | 10 | 2.0 | 1.9 |
| Benzo[k]fluoranthene [ppb] | 1.7 | n.d. (<0.5) | n.d. (<0.5) |
| Total amount of PAHs [ppb] | 16.5 | 7.4 | 6.2 |

n.n. = not detectable

The PAHs phenanthrene, anthracene, benz[a]anthracene, chrysene, benzo[b]fluoranthene, benzo[a]pyrene, indeno[1,2,3-cd]pyrene, dibenzo[ah]anthracene and benzo[ghi]perylene which are not cited in the table, were not detectable in the extracts according to Comparative Example 1 and Examples 1 and 2 (content <0.5 ppb).

1-12. (canceled)

13. A method for preparing an extract from Ginkgo biloba having a reduced content of polycyclic aromatic hydrocarbons, the method comprising:
(a) extracting fresh or dried green leaves of Ginkgo biloba (drug) at a temperature of about 40 to 100° C. using aqueous acetone, an aqueous alkanol having 1 to 3 carbon atoms or anhydrous methanol,
(b) separating the organic solvent from the extract to a maximum content of 2% by weight, wherein water may be added during the final distillation steps,
(c) diluting the remaining concentrated aqueous solution with water to a solids content of 5 to 25% by weight, cooled under agitation to a temperature below 6° C., allowed to stand for at least 1 hour at this temperature and the resulting precipitate comprising lipophilic components which are not well soluble in water, is removed,
(d) adding ammonium sulfate to the remaining aqueous solution and the solution formed is extracted with methyl ethyl ketone or a mixture of methyl ethyl ketone and acetone,
(e) concentrating the extract obtained to a solids content of 50 to 70% by weight and the concentrate thus obtained is diluted with water and ethanol such that a solution containing 50% by weight water and 50% by weight ethanol at a solids content of about 10% by weight is obtained,
a filtration is carried out and the filtrate is further processed in (f),
(f) adding an aqueous solution of a lead salt to the solution thus obtained until a change in colour from brown to amber occurs, and the precipitate formed is removed, or a polyamide is used instead of the lead salt,
(g) extracting the remaining aqueous alcoholic solution with heptane in order to further remove the alkylphenol compounds,
(h) concentrating the remaining aqueous alcoholic solution under reduced pressure to an ethanol content of about 5% by weight and added with ammonium sulfate to a content of 20% by weight,
(i) extracting the solution obtained with a mixture of methyl ethyl ketone and ethanol in a ratio of 8:2 to 5:5 (w/w),
(k) drying the resulting organic phase with ≦20% by weight ammonium sulfate and concentrated to a solids content of 50 to 70% by weight, added with ethanol such that an ethanol content of at least 80% by weight is obtained, maintained for at least 2 h at ≦12° C. and filtered,
(l) concentrating the resulting filtrate under reduced pressure at a maximum temperature of 60 to 80° C. and dried, thereby obtaining a dry extract having a water content of less than 5% by weight.

14. A method of claim 13 wherein in step (a) the temperature is 40 to 60° C. and/or the drug-to-solvent ratio is 1:4 to 1:20.

15. A method of claim 13 wherein in step (b) the organic solvent is separated from the extract to a maximum content of 1% by weight.

16. A method of claim 13 wherein in step (c) allowing to stand is carried out for at least 10 hours at below 6° C.

17. A method of claim 13 wherein in step (d) about 30% by weight ammonium sulfate is added to the remaining aqueous solution and/or the solution formed is extracted with a mixture of methyl ethyl ketone and acetone in a ratio of 6:4 (w/w).

18. A method of claim 13 wherein the lead salt in step (f) is lead hydroxide acetate.

19. A method of claim 13 wherein the extraction in step (g) is carried out at least five times.

20. A method of claim 13 wherein in step (i) the solution obtained is extracted with a mixture of methyl ethyl ketone and ethanol in a ratio of 6:4 (w/w).

21. An extract obtainable from Ginkgo biloba having a reduced content of polycyclic aromatic hydrocarbons which is obtainable by a method of claim 13.

22. An extract of claim 21 wherein the extract has a content of polycyclic aromatic hydrocarbons of ≦10 ppb.

23. A method of treating a subject suffering from or susceptible to dementia and one or more symptoms thereof and/or a cerebral or peripheral blood circulation disorder, comprising administering to the subject an extract of claim 21.

24. A method of claim 23 wherein the subject is suffering from dementia and one or more symptoms thereof and/or a cerebral or peripheral blood circulation disorder.

25. A method of claim 23 wherein the subject is suffering from dementia.

26. A method of claim 23 wherein the subject is suffering from a cerebral or peripheral blood circulation disorder.

27. A method of claim 23 wherein the subject is identified as suffering from dementia and one or more symptoms thereof and/or a cerebral or peripheral blood circulation disorder and the extract is administered to the identified subject.

28. A medicament, food product or other preparation comprising an extract of claim 21.

29. A method for preparing an extract from Ginkgo biloba having a reduced content of polycyclic aromatic hydrocarbons, the method comprising:
(a) extracting fresh or dried green leaves of Ginkgo biloba (drug) at a temperature of about 40 to 100° C. using aqueous acetone, an aqueous alkanol having 1 to 3 carbon atoms or anhydrous methanol,
(b) separating the organic solvent from the extract to a maximum content of 2% by weight,
(c) diluting the remaining concentrated aqueous solution with water to a solids content of 5 to 25% by weight, cooling under agitation to a temperature below 6° C., and removing the resulting precipitate comprising lipophilic components,
(d) adding ammonium sulfate to the remaining aqueous solution and the solution formed is extracted with methyl ethyl ketone or a mixture of methyl ethyl ketone and acetone,
(e) concentrating the extract obtained to a solids content of 50 to 70% by weight and the concentrate thus obtained is diluted with water and ethanol,
(f) adding an aqueous solution of a polyamide or lead salt to the solution thus obtained and the precipitate formed is removed,
(g) extracting the remaining aqueous alcoholic solution to further remove the alkylphenol compounds,
(h) concentrating the remaining aqueous alcoholic solution under reduced pressure,
(i) extracting the solution obtained with a mixture of methyl ethyl ketone and ethanol in a ratio of 8:2 to 5:5 (w/w),
(k) drying the resulting organic phase with ≦20% by weight ammonium sulfate and concentrated to a solids content of 50 to 70% by weight, added with ethanol such that an ethanol content of at least 80% by weight is obtained, maintained for at least 2 h at ≦12° C. and filtering,
(l) concentrating the resulting filtrate under reduced pressure at a maximum temperature of 60 to 80° C. and dried, thereby obtaining a dry extract having a water content of less than 5% by weight.
